(19) United States
(12) Patent Application Publication
(10) Pub. No.: US 2018/0203001 A1
(43) Pub. Date: Jul. 19, 2018

(54) METHODS OF DETERMINING URINARY CALCULI COMPOSITION

(71) Applicant: LONDON HEALTH SCIENCES CENTRE RESEARCH INC., London (CA)

(72) Inventors: Hon Sing LEONG, Edmonton (CA); Andre ST. AMANT, Zurich (CA)

(21) Appl. No.: 15/871,750

(22) Filed: Jan. 15, 2018

Related U.S. Application Data
(60) Provisional application No. 62/446,148, filed on Jan. 13, 2017.

(51) Int. Cl. G01N 33/53

(52) U.S. Cl. CPC G01N 33/5308; G01N 2800/345; G01N 2800/50; G01N 2800/52

(57) ABSTRACT

A method of detecting urinary calculi in a subject. Detecting urinary calculi includes first exposing the urine sample to at least one labeled binding probe that is specific to calcium/magnesium-based urinary calculi and second detecting urinary calculi bound to the at least one labeled binding probe. Methods for measuring, monitoring and/or determining subtype of urinary stone composition in a subject are also provided.

### CROSS REFERENCE TO RELATED APPLICATIONS

This application claims priority from U.S. Provisional Ser. No. 62/446,148, filed Jan. 13, 2017, the content of which is hereby incorporated by reference into the present application.

### FIELD OF THE INVENTION

The present invention relates to methods of determining urinary calculi composition.

### BACKGROUND OF THE INVENTION

Nephrolithiasis is a common and often painful urological disorder, with the lifetime risk estimated to be 10-15% in the United States [1]. In a report by the National Health and Nutritional Examination Survey (NHANES), the prevalence of kidney stone disease has increased from 3.8% in the 1970's to 8.4% in 2010 [2]. The risk for forming renal stones is primarily influenced by urinary composition, which in turn is linked to metabolic imbalances and certain lifestyle choices.

Current guidelines for patient workup begin with a complete history to identify risk factors, followed by laboratory investigations and where a stone is available, to determine its composition [3]. A non-contrast helical CT scan with 5 mm cuts remains the gold standard for diagnosing renal stones, with subsequent urinalysis, serum electrolytes, and renal function tests providing additional information [4-6]. Currently there are no non-invasive methods to accurately determine stone composition. Two or more 24-hour urine samples tested for metabolic abnormalities such as hypercalciuria, hyperoxaluria, hyperuricosuria and hypocitriuria, may hint at the underlying stone type [7]. However, these techniques are not accurate and are even misleading in some cases. For example, the 24-hour urine analysis is subject to daily variations in the patient's dietary and fluid intake, which can skew urinary composition [8]. Non-contrast helical CT-Scans, although very sensitive and specific for identifying calculi location, rarely provide definitive information on calculi composition 9. Stone size does not correlate well with stone type, and Hounsfield units, which measure the relative density of an object on a CT-scan, cannot accurately predict stone type [10,11]. Dual-energy CT-scan marginally improves accuracy in predicting stone type [12].

In cases where a stone is available for analysis, multiple methods are used for identifying stone composition including chemical analysis, x-ray diffraction and infrared spectroscopy which is considered the laboratory gold standard [13]. Results usually vary due to differences in instrumentation and the procedure by which the stone is sampled and processed. Therefore, there is the potential for error in reporting calculi composition [14]. In a study of 25 stones, composition was initially measured by micro CT-scan and then sent to multiple commercial laboratories for analysis with the highest accuracy observed in stones composed of only one element and incorrect diagnosis in 50% of struvite stones and 20% of stones that contained apatite [14].

A non-invasive, inexpensive, and high-throughput test for determining urinary stone composition would significantly improve the clinical management of patients with new or recurring stones.

### SUMMARY OF THE INVENTION

The present invention is broadly summarized as relating to methods of detecting urinary calculi or particles, measuring the amount of urinary calculi or particles and/or determining urinary calculi or particles composition.

In an aspect, a method for detecting urinary calculi/particles in a subject is provided. Detecting calculi comprises first exposing a urine sample obtained from the subject to at least one labeled binding probe that is specific to calcium/magnesium-containing urinary calculi and second detecting urinary calculi bound to the at least one labeled binding probe.

In one embodiment, the detection of the at least one labeled binding probe is obtained with one or more of stereoscopic, microscopic and/or flow cytometry analysis of the urine sample. In some embodiments, flow cytometry is nanoscale flow cytometry.

In another aspect, a method for determining urinary calculi/particles type in a subject is provided. Determining the composition of calculi/particles comprises first exposing a urine sample obtained from the subject to at least one labeled binding probe that is specific to calcium/magnesium—containing urinary calculi and second detecting urinary calculi bound to the at least one labeled binding probe, wherein the detected urinary calculi bound to the at least one labeled binding probe are calcium based urinary calculi.

In one embodiment, the detection of the at least one labeled binding probe is obtained with one or more of stereoscopic, microscopic, and/or flow cytometry analysis of the urine sample. In some embodiments, flow cytometry is nanoscale flow cytometry.

In an aspect, a method for measuring the amount urinary calculi or particles in a subject is provided. The method comprises measuring the amount of calculi in a urine sample obtained from the subject. Measuring the amount of calculi comprises first exposing the urine sample to at least one labeled binding probe that is specific to calcium/magnesium-containing urinary calculi and second measuring the amount of urinary calculi bound to the at least one labeled binding probe.

In one embodiment, the measured amount of urinary calculi bound to the at least one labeled binding probe is then compared with a reference value and diagnosis of the subject is based on results of the comparison.

In some embodiments, the measurement of calculi bound to the at least one labeled binding probe is obtained by flow cytometry. In some embodiments, flow cytometry is nanoscale flow cytometry.

In another aspect, the present invention provides for a method for monitoring a therapeutic treatment to reduce or eliminate urinary calculi or particles in a subject, the method comprising:

a) measuring the amount of urinary calculi in a first urine sample obtained from the subject at a first time point, the measuring comprising:
i) exposing or contacting the first urinary sample to at least one labeled binding probe that is specific to calcium/magnesium-containing urinary calculi; and
ii) measuring the amount of urinary calculi bound to the at least one labeled binding probe;
b) measuring the amount of urinary calculi in a second urine sample obtained from the subject at a subsequent time point, the measuring comprising:
i) exposing the second urine sample to at least one labeled binding probe that is specific to calcium/magnesium-containing urinary calculi; and
ii) measuring the amount of urinary calculi bound to the at least one labeled binding probe in the second urine sample;
c) comparing the measured urine calculi bound to the at least one labeled binding probe in the second urine sample with the measurement obtained in step (a) (ii); and
d) detecting increased or reduced urinary calculi in the subject when there is a difference in the measurement obtained in step (a) (ii) relative to the measurement obtained in step (b) (ii).

In embodiments of the present invention, the first time point is a time point before the start of the treatment or during the treatment.

In embodiments of the present invention, the second time point is a time point during the treatment or after conclusion of the treatment.

In one embodiment, the measured amount of urinary calculi bound to the at least one labeled binding probe is then compared with a reference value and diagnosis of the subject is based on results of the comparison.

In some embodiments, the measurement of calculi bound to the at least one labeled binding probe is obtained by flow cytometry. In some embodiments, flow cytometry is nanoscale flow cytometry.

In an aspect, the present invention provides of a method of carrying out a test for the detection of particles in urine to determine the susceptibility of a subject for kidney stones whereby a urine sample is obtained from the subject, exposing the urine sample to at least one labeled binding probe that is specific to calcium/magnesium-containing urinary particles and detecting urinary particles bound to the at least one labeled binding probe. The detection of calcium-rich particles in the urine being indicative of the subject's susceptibility for kidney stones.

In one embodiment, the detection of the at least one labeled binding probe is obtained with one or more of stereoscopic, microscopic and/or flow cytometry analysis of the urine sample. In some embodiments, flow cytometry is nanoscale flow cytometry.

The at least one labeled binding probe according to any of the above aspects and embodiments includes detectably-labeled Alendronate.

The detectable label of any of the above aspects and embodiments includes a fluorophore. In some embodiments, the fluorophore is FITC or Cy5.

### BRIEF DESCRIPTION OF THE DRAWINGS

FIG. 1 Macroscopic Analysis of Calculi Subtypes with Fluorescent Alendronate Probes. (A) Macroscopic images of alendronate treated renal calculi of different compositions. Brightfield images are compared to fluorescein channel images. Fluorescein fluoresces green where present; denoted as FITC in figures. (B) Microscopic images of alendronate treated renal calculi fragments of varying compositions. (C) Microscopic images of notdronate treated renal calculi fragments of varying compositions. Uric acid auto fluorescence or unspecified binding is suggested. (D) Macro scale images of alendronate and notdronate treated renal calculi fragments of varying compositions in PBS. Sediment dyed blue indicates presence of Cy5 fixed alendronate or notdronate.

FIG. 2 Macroscopic Analysis of Calculi Subtypes of Various Heterogeneity with Fluorescent Alendronate Probes. (A) Macro scale images of alendronate treated renal calculi of varying calcium-based compositions. Bright field images are compared to fluorescein channel images; denoted as FITC in figures. (B) Microscopic images of alendronate treated renal calculi fragments of varying calcium based compositions. (C) Microscopic images of notdronate treated renal calculi fragments of varying calcium based compositions. Fluorescein channel images that fluoresce green suggest auto-fluorescence or unspecific binding. (D) Macro scale images of alendronate and notdronate treated renal calculi fragments of different calcium based compositions in PBS. Sediment dyed blue indicates presence of Cy5 fixed alendronate or notdronate.

FIG. 3 Nanoscale Flow Cytometry of Calculi Nanocrystals in Saline. (A) PBS is run through a nanoscale flow cytometer as a negative control. Fluorescent signal as well as Large/Small Angle Light Scattering (LALS/SALS) is recorded. Fluorescent signal is characterised by a dual positive reading of Fluorescein (denoted as FITC) and Cy5, whereas LALS/SALS signifies size of particles. (B) Hydroxyapatite (HA) nanoparticles suspended in PBS used as a positive control to renal calculi fragments. (C) Alendronate treated renal calculi fragments suspended in PBS. (D) Notdronate treated renal calculi fragments suspended in PBS. Signal quantifies non-specificity of binding.

FIG. 4 Nanoscale Flow Cytometry of Calculi Nanocrystals in Healthy Volunteer Urine. (A) Healthy volunteer urine sonicated and ran through a nanoscale flow cytometer. (B) Healthy volunteer urine is treated with renal calculi fragments, sonicated and ran through a nanoscale flow cytometer. (C) Healthy volunteer urine treated with renal calculi fragments as well as Alendronate and sonicated before being run through a nanoscale flow cytometer. (D) Healthy volunteer urine is treated with renal calculi fragments as well as Notdronate and then sonicated and analyzed by nanoscale flow cytometry.

FIG. 5 Nanoscale Flow Cytometric Analysis and Enumeration of Alendronate +ve Nanocrystals in Patient Urine Samples. Analysis of patient urine samples by nanoscale flow cytometry with alendronatefluorescein (denoted as FITC in figure) probe revealed the highest nanocrystal count (±SEM) in calcium oxalate and brushite calculi patient cohorts (A). * denotes p<0.05, one-way ANOVA with Bonferroni correction. A positive correlation was observed when nanocrystal counts in urine were plotted against calcium oxalate calculi mass using non-parametric rank correlation, revealing a correlation coefficient of R2=0.51, p=0.089 (B). (C) Representative brightfield, birefringence, and fluorescence images of alendronate-fluorescein stained (FIG. 5C) and notdronate-fluorescein stained patient urine samples (D), revealing an abundance of fluorescein-positive nanocrystals in calcium oxalate and brushite calculi urine samples.

FIG. 6 Re-evaluation of Specific Calculi with High Alendronate +ve Nanocrystal Counts in Patient Urine. (A, B) A single uric acid calculus with a high urine nanocrystal count was pulverized and stained with probe, revealing an abundance of alendronate +ve fragments within the uric acid calculi. Calculi fragments also exhibited birefringent signal. (C, D) A single struvite calculi with a high urine nanocrystal count was pulverized and stained with probe, revealing an abundance of alendronate +ve fragments within the struvite calculi. Calculi fragments did not exhibit birefringent signal. Scale bars are 0.5 mm.

FIG. 7 Analysis of Calculi Subtypes using Petrographic Thin Sectioning. (A) Microscopic bright field images of a number of petrographic thin sections of calculi with supposed varying compositions. (B) Microscopic birefringent images of a number of calculi with supposed varying compositions. Vibrant, colorful images indicate presence of calcium-based crystals. (C) Alendronate treated petrographic thin sections. (D) Notdronate treated petrographic thin sections. Uric acid auto fluorescence or unspecified binding is suggested.

### DESCRIPTION OF THE INVENTION

### Definitions

Unless defined otherwise, all technical and scientific terms used herein have the same meaning as commonly understood by one of ordinary skill in the art to which this invention belongs. Also, unless indicated otherwise, except within the claims, the use of “or” includes “and” and vice versa. Non-limiting terms are not to be construed as limiting unless expressly stated or the context clearly indicates otherwise (for example “including”, “having” and “comprising” typically indicate “including without limitation”). Singular forms including in the claims such as “a”, “an” and “the” include the plural reference unless expressly stated otherwise. In order to aid in the understanding and preparation of the within invention, the following illustrative, non-limiting, examples are provided.

The term “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the enumerated value.

As used herein, the terms “diagnose”, “diagnosing” and “diagnostic” refer to the process of determining a disease state or disorder in a subject. In determining disease state a diagnostician might classify one or more characteristics of a subject, such as, for example, symptoms and/or biomarkers. A “diagnostic assay” is referred to herein as a tool that a diagnostician might use to narrow the diagnostic possibilities.

As used herein, the term “subject” refers to a mammal, such as, for example, a human, non-human primate, mouse, rat, dog, cat, horse, or cow. In some embodiments, a subject is human and might be referred to as a patient. A subject can be one who has been previously diagnosed or identified as having a disease, and optionally one who has already undergone, or is undergoing, a therapeutic intervention for a disease. Alternatively, a subject can also be one who has not been previously diagnosed as having a disease.

As used herein, the terms “detect”, “detection” and “detecting” refer to a quantitative or qualitative determination of a property of an entity, for example, quantifying the amount or concentration of a molecule or the activity level of a molecule. The term “concentration”, “amount” or “level” can refer to an absolute or relative quantity. Measuring a molecule may also include determining the absence or presence of the molecule. Various methods of detection are known in the art, for example fluorescence analysis. In this regard, biomarkers can be measured using fluorescence detection methods, or other methods known to the skilled artisan.

As used herein, the term “reference value” refers to a baseline value. A baseline value represents the number of calculi, also referred to as particles, in a urine sample from an effective number of subjects who do not have the disease of interest and who are positive for the biomarker of interest. A reference value can also comprise the number of urinary calculi in a urine sample from an effective number of subjects who have the disease of interest, as confirmed by an invasive or non-invasive technique.

As used herein, the terms “indicative of”, “associated with” and “correlated to” refer to the determination of a relationship between one type of data with another or with a state. In some embodiments, correlating the measurement with disease comprises comparing the number of urinary calculi positive for a biomarker with a reference value. In some embodiments, correlating the measurement with disease comprises determining the subject's disease state.

As used herein, the terms “treatment”, “treatment regimen”, “therapy” and “therapeutic treatment” refer to an attempted remediation of a health problem. In some embodiments, treatment can be selected from, administering a disease-modulating drug to a subject, administering disease-modulating radiation to a subject, surgery or scheduling for a further appointment with a medical practitioner. Treatment refers to initiating therapy, continuing therapy, modifying therapy or ending therapy.

As used herein, the terms “prophylaxis” and prophylactic” refer to measures taken to prevent disease. Prophylactic treatment includes, for example, measures to reverse, prevent or slow down physiological features that are precursors to disease.

As used herein, the term “binding probe” refers to compounds that are used to detect the presence of, or to quantify, relatively or absolutely, a target molecule or target sequence and that will bind to the target molecule or sequence, either directly or indirectly. Generally, the binding probe allows attachment of a target molecule or sequence to the probe for the purpose of detection. In some embodiments, the target molecule or sequence is a biomarker. It follows that the composition of the binding probe will depend on the composition of the biomarker. Binding probes for a variety of biomarkers are known or can be generated using known techniques. For example, when the biomarker is a protein, the binding probes include for example, small molecules and proteins.

As used herein, the terms “label” and “labeled” refer to a composition detectable by spectroscopic, photochemical, biochemical, immunochemical, chemical, or other physical means. A compound that is labeled has at least one molecule, element, isotope or chemical compound attached to it to enable the detection of the compound. For example, useful labels include fluorescent dyes, which might also be referred to as fluorophores.

As used herein, the term “fluorophore” refers to a molecule or part of a molecule that absorbs energy at one wavelength and re-emits energy at another wavelength. Detectable properties of fluorophores include fluorescence intensity, fluorescence lifetime, emission spectrum characteristics, energy transfer, and the like. Fluorophores are of use in various embodiments of the present invention, at least due to their strong signals, which provide a signal-to-noise ratio sufficient to allow interpretation of the signals. Suitable fluorophores include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue, Texas Red, Alexa dyes and others described in the 6th Edition of the Molecular Probes Handbook by Richard P. Haugland.

As used herein the term “negative control” refers to an element or group used in an experiment to ensure that a negative result is produced when a negative result is expected. For example, a negative control binding probe, as referred to herein, is a probe that should not bind to hydroxyapatitie being examined because the probe's component that is responsible for specific binding is not present in the sample being examined. Thus, when assayed, if a negative control binding probe successfully binds to a sample, then it can be inferred that a confounding variable acted on the experiment, suggesting that the positive results are likely not due the intended specific binding.

As used herein, the term “monitoring” refers to the observation of a disease over time. Monitoring of a subject's disease state can be performed by continuously measuring certain parameters and/or by repeatedly performing a medical test. In some embodiments of the present invention, a subject's disease state is monitored by repeatedly obtaining bodily fluid samples such as urine, assaying the samples using the method disclosed herein and comparing the results of the assays with one another and/or with a reference value to identify any change in the subject's disease state.

As used herein, the term “kit” refers to a collection of elements that together are suitable for a defined use.

“Calcium/magnesium-containing urinary calculi”: calcium-containing, magnesium-containing and/or calcium and magnesium-containing urinary calculi.

Overview

The present invention is based on the inventor's hypothesis that that because renal calculi form in turbulent conditions throughout the urinary tract, they release a range of macroscopic and microscopic calculi-derived fragments into the urine. The inventor further hypothesized that these fragments can be analysed using probes specific for calcium [15] and high throughput analysis techniques such as nanoscale flow cytometry. The inventor has shown that urine-based analyses focused on enumerating nanocrystals released by the calculi into the urine milieu reveals excellent agreement with the internal composition of the calculi as determined by petrographic thin section analysis.

To confirm composition throughout the entire stone, the inventor used petrographic thin sectioning to expose the internal structure of the stones for histological analysis. The inventor has repurposed the bisphosphonate Alendronate for conjugation to fluorescent probes [16] such as Fluorescein and Cy5 for nanoscale flow cytometry of urine [17] and histological analysis of petrographic thin sections of renal calculi. Nanoscale flow cytometry can analyze particles that exhibit a size diameter range between 100-1000 nm and is equipped with three lasers (405 nm, 491 nm, 643 nm) and five detectors for multi-parametric analysis of events in complex mixtures such as plasma or urine. Petrographic thin sectioning has been previously used in the examination of renal calculi [18-21].

Without further elaboration, it is believed that one of ordinary skill in the art can, based on the description presented herein, utilize the present invention to the full extent. All publications cited are incorporated by reference.

Some embodiments involve a method for diagnosing or determining stone burden and subtype in a subject. The method comprises obtaining a urine sample from the subject.

The urine sample is then analyzed, for example by a flow cytometry assay, to specifically detect nanocrystals that bind fluorescent alendronate probes, fluorescently-labeled binding probes specific to calcium/magnesium-containing urinary calculi. In some embodiments, flow cytometry is carried out using a nanoscale flow cytometer.

In some embodiments, the fluorescently-labeled binding probe specific to calcium/magnesium-containing urinary calculi is Alendronate-FITC or Alendronate-Cy5. Alendronate is a bisphosphonate that specifically binds to HA in bone tissue. Detection of microparticles containing HA allows for specific identification of a sample containing HA.

In some embodiments, the nanocrystal count identified in a sample by the flow cytometry assay is compared with a reference value.

The reference value can be a baseline number that represents the amount of nanocrystals that are found in a given volume of urine sample from a typical subject who has a healthy urinary calculi state. Where a reference value is indicative of a healthy state, a measured value in a subject that is greater than said reference value would be indicative of unhealthy count of nanocrystals in the subject. It is also contemplated herein that a reference value could, in contrast, represent the amount of nanocrystals that are found in a given volume of sample from a subject having unhealthy urinary calculi state. Where such a reference value is used, a measured value in a subject that is less than said reference value would be indicative of a healthy state in the subject; a measured value in a subject that is greater than or equal to said reference value would be indicative of unhealthy urinary calculi composition in the subject.

In some embodiments, the flow cytometry assay comprises exposing a urine sample to a composition comprising at least one labeled binding probe that is specific to calcium/magnesium-containing urinary calculi, for example, Alendronate. In some embodiments, the at least one binding probe is labeled with a fluorophore. In some embodiments, the composition comprises two differently labeled binding probes specific to calcium/magnesium-containing urinary calculi. In some embodiments, the labels are fluorophores. When selecting suitable fluorophores the excitation wavelength of the fluorophore conjugated to the first of the two binding probes should be distinct from the excitation wavelength of the fluorophore conjugated to the second of the two binding probes.

Suitable fluorophores include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue, Texas Red, Alexa dyes and others described in the 6th Edition of the Molecular Probes Handbook by Richard P. Haugland. In some embodiments, Cyanine-5 (Cy5) is conjugated to the first binding probe and flourescein isothiocyanate (FITC) is conjugated to the second binding probe. Use of two fluorescent binding probes specific to to calcium/magnesium-containing urinary calculi is preferable, at least because it allows for detection of non-specific binding of fluourescently-labeled probes.

In some embodiments, negative controls are used in the method of determining urinary calculi composition, to allow for quantification of microparticles that are positive for calcium and/or magnesium.

The inventor synthesized a suitable negative control, which is a fluorescently-labeled molecule referred to herein as “NOT-dronate”, wherein NOT-dronate is represented by formula (I):

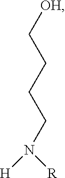

wherein R is a fluorophore. In some embodiments, the first negative control is NOT-dronate-Cy5 and the second negative control is NOT-dronate-FITC.

In some embodiments of the method, a portion of the sample of the bodily fluid is removed and exposed to a composition comprising the first and second negative control binding probes, such as NOT-dronate-Cy5 and NOT-dronate-FITC. The exposed sample is then analyzed by a flow cytometry assay to specifically detect microparticles bound to fluorescent labels in the bodily fluid sample. If any microparticles are found to bind to one or more of the negative control probes, then a confounding variable might be responsible for any fluorescent microparticles that are identified in the disclosed assay for detecting bone microparticles. If the fluorescence of the negative control probes is not observed, then confounding variables can be eliminated as possible cause for positive results that are found in the disclosed assay for detecting calcium/magnesium-containing urinary calculi.

In some embodiments, a method of monitoring the efficacy of a therapy to reduce urinary calculi is provided. In some monitoring methods, a first urine sample is obtained from the subject at a first time point, for example before therapeutic treatment starts. The first sample is then subjected to analysis comprising measurement of the amount of urinary calculi in the sample. The monitoring method can further comprise, obtaining a second urine fluid sample from the subject at a second time point subsequent to the first time point, for example, after treatment started. The second sample is then subjected to analysis comprising measurement of the amount of urinary calculi in the sample. The measurement obtained from the second sample is compared to the measurement obtained from the first sample to determine if the subject's disease state has improved, worsened or remained constant since the first time point. A treatment regimen can then be effectuated based on the subject's disease state. The treatment might involve, for example, drug, nutrient supplement or lifestyle intervention or it might involve further monitoring.

In some embodiments, a kit is provided for detecting the amount of urinary calculi in a urine sample. The kit comprises a first labeled isotype negative control for labeled Alendronate, Alendronate being specific to calcium/magnesium-containing urinary particles. The kit may further comprise a first labeled binding probe specific to calcium/magnesium-containing urinary particles, such as Alendronate. In some embodiments, the first labeled binding probe is Alendronate-Cy5. In some embodiments, the first isotype negative control is NOT-dronate-Cy5.

In some embodiments, the kit further comprises a second labeled binding probe specific to calcium and/or magnesium-containing urinary particles, and a second isotype negative control for the second labeled binding probe specific to calcium and/or magnesium-containing urinary particles. In some embodiments, the second labeled binding probe is Alendronate-FITC. In some embodiments, the second isotype negative control is NOT-dronate-FITC.

In some embodiments, the kit provides the first and second binding probes in a first sealed container. In some embodiments, the negative controls are provided in a second sealed container.

In some embodiments, the kit might comprise a carrier, such as a box, carton, tube or the like, having disposed therein one or more sealed containers, such as vials, tubes, ampoules, bottles, pouches, envelopes and the like. In some embodiments, the kit might comprise one or more media or media ingredients or reagents for the measurement of the various biomarkers disclosed herein. For example, kits may also comprise, in the same or different containers, one or more suitable buffers or probes. The kits may also comprise one or more instructions or protocols for carrying out embodiments of the present invention.

### Examples

Materials and Methods

Stone and Urine Sample Collection. All experiments and sample collection procedures were approved by The University of Western Ontario REB panel. Informed consent was obtained from all patients prior to surgery and collection of stone/urine samples. All stone and urine samples were collected from enrolled patients who underwent Percutaneous Nephrolithotomy (PCNL) for renal stone disease. Patients with end stage renal disease (ESRD) and/or tumors were excluded from the study. Of the 31 patients enrolled in the study, 1 had a history of hyperparathyroidism with no biochemical or clinical features at the time of the study. None of the patients were hyperuricemic. 8 patients were taking vitamin D/calcium supplements (Table 1). 10 mL of the patient's bladder urine was collected in a sterile fashion and stored at −80° C. Stone samples were stored at −20° C. Separate fragments from all stone samples underwent infrared spectroscopy based analysis at the hospital laboratory for initial evaluation of stone composition (Table 2).

TABLE 1
Clinical and radiological characteristics of the 31 patients

| | | |
|---|---|---|
| Mean age (years | 49.3 | (19-89) |
| Women | 17/31 | (54.8%) |
| BMI (kg/m2) | 28.7 | (17.4-43.5) |

Comorbidities

| | | |
|---|---|---|
| Hypertension | 13/31 | (41.9%) |
| Type 2 diabetes | 7/31 | (22.6%) |
| Recurrent stone formers | 2/31 | (6.5%) |
| Hyperparathyroidism | 1/31 | (3.2%) |
| Hyperuricemia | 0/31 | (0%) |
| Vitamin D/Calcium Supplement Use | 8/31 | (25.8%) |
| Multiple stones in preoperative | 19/31 | (61.3%) |

| | |
|---|---|
| Mean largest diameter (mm) | 24.7 |
| Mean Hounsfield Units | 905 |
| Mean standard deviation (HU) | 159 |

TABLE 2
Hospital laboratory stone composition analysis

| | | n |
|---|---|---|
| Single composition | | 13 |
| | Uric acid | 4 |
| | COM | 3 |
| | Brushite | 3 |
| | Apatite | 2 |
| | Cystine | 1 |
| Multiple composition | | 18 |
| | COM + COD | 7 |
| | COM + COD + Apatite | 5 |
| | COM + Apatite | 2 |
| | Struvite + Apatite | 2 |
| | COM + Uric Acid | 1 |
| | COM + Apatite + Struvite | 1 |

Alendronate and Notdronate Probe Synthesis.

Fluorescent analysis of stone composition was carried out using alendronate, a bisphosphonate with a high affinity to bind to hydroxyapatite. Alendronate was conjugated with NHS-fluorescein and Cy5 fluorescent markers. As a negative control, the novel compound Notdronate of the present invention was used. Notdronate is similar in structure to alendronate but lacks the bisphosphonate functional group, which prevents it from binding to hydroxyapatite. Notdronate was also conjugated to fluorescein isothiocyanate and Cy5.

Synthesis of Alendronate-Fluorescin and Notdronate-Fluorescin

01-051A (Alendronate-FITC):

Sodium alendronate (34.0 mg, 106 μmol) was dissolved in saturated NaHCO_{3 (aq)} (1 mL). Fluorescein (5/6) NHS ester (10 mg, 21 μmol) dissolved in DMF (100 μL) was added and the solution stirred for 2 days in the dark. The product was dried, suspended in H₂O (1 mL) and dialyzed (cellulose ester, MWCO 0.1-0.5 kD) with water (3×500 mL). The final product's concentration was determined by the UV absorption (ε_{493 nm}=70,000 M⁻¹ cm⁻¹). The solution was subjected to RP-FCC (Isolera One, Biotage KP-C18-HS 12 g cartridge) with a gradient from 0 to 30% MeOH in H₂O. The product was lyophilized to yield FITC alendronate (8.6 μmol, 41%) as an orange powder. UP LC-MS (waters) method: 5-40% acetonitrile in water, both contain 0.1% formic acid, 3 mins run; Calculated m/z 608.07 (MH+), Found m/z: 608.10; RT (min) 1.40. Purity: >95%.

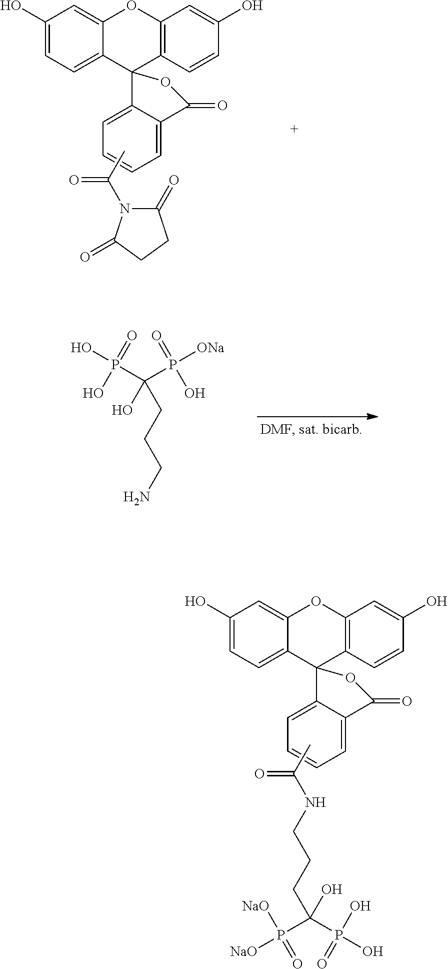

01-051B (Notdronate-FITC)

4-Amino-1-butanol (20 mg, 200 μmol) was dissolved in saturated NaHCO_{3 (aq)} (1 mL). Fluorescein (5/6) NHS ester (10 mg, 21 μmol) dissolved in DMF (100 μL) was added and the solution stirred for 2 days in the dark. The reaction mixture was subjected to RP-FCC (0 to 100% MeOH in H₂O) and the final product concentration was determined by the UV absorption (ε493 nm=70,000 M⁻¹ cm⁻¹). The solution was subjected to RP-FCC (Isolera One, Biotage KP-C18-HS 12 g cartridge) with a gradient from 0 to 25% MeOH in H₂O. The product was lyophilized to yield fluorescein-4-butanol (4.8 μmol, 23%) as an orange powder. UP LC-MS (waters) method: 5-40% acetonitrile in water, both contain 0.1% formic acid, 3 mins run; Calculated m/z 448.14 (MH⁺), Found m/z: 448.04; RT (min) 2.23. Purity: >95%.

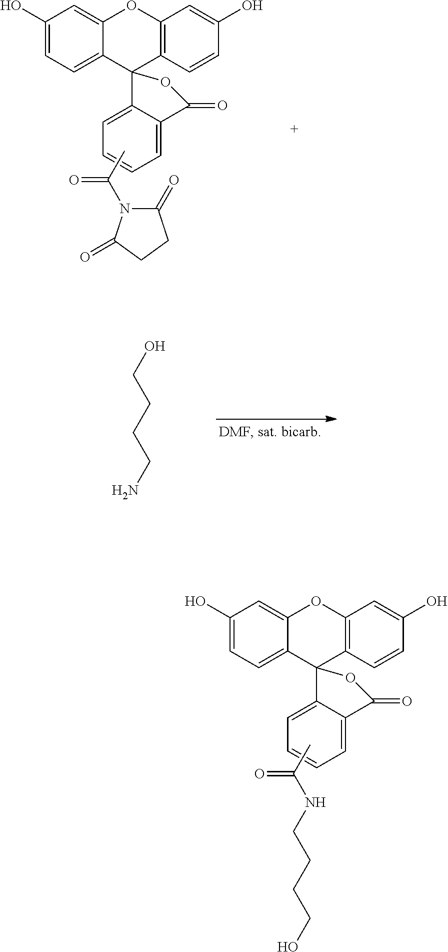

Synthesis of Alendronate-Cy5 and Notdronate-Cy5

AS-01-077A (Aldronate-Cy5)

Sodium alendronate (34.0 mg, 125 μmol) was dissolved in 0.1 M NaHCO_{3 (aq)} (1 mL). Sulfo-Cy5 NHS ester (12.5 mg, 16.4 μmol) dissolved in DMF (125 μL) was added and the solution stirred overnight in the dark. The reaction mixture was dialysed (cellulose ester, MWCO 0.1-0.5 kD) with water (4×500 mL with water change at 2h, 4h, 6h and dialysed overnight). The solution was subjected to RP-FCC (Isolera One, SiliaSep™ C18 12 g cartridge) with a gradient from 0 to 100% MeOH in H₂O. The fractions were lyophilized to yield Sulfo-Cy5 alendronate (0.94 μmol, 6%) as a blue powder. UP LC-MS (waters) method: 5-90% acetonitrile in water, both contain 0.1% formic acid, 3 mins run; Calculated m/z 874.22 (MH⁺), Found m/z: 874.18; RT (min) 0.82. Purity: >95%.

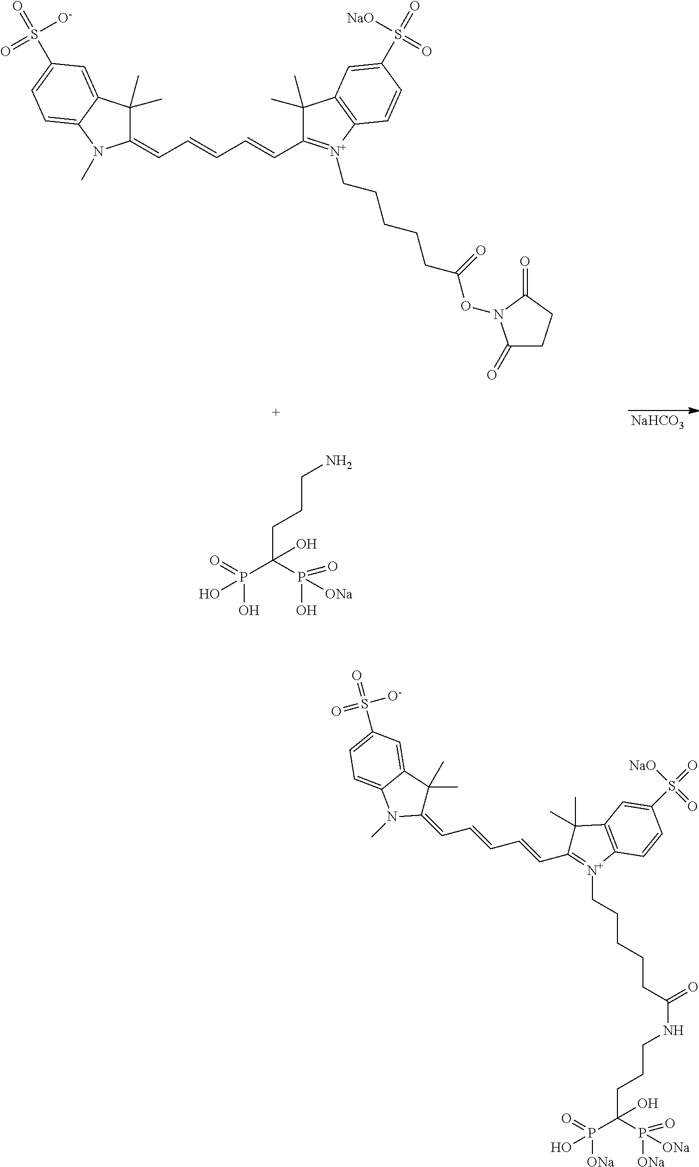

AS-01-077B (Notdronate Cy5)

4-Amino-1-butanol (20 mg, 200 μmol) was dissolved in 0.1 M NaHCO_{3 (aq)} (1 mL). Sulfo-Cy5 NHS ester (12.5 mg, 16.4 umol) dissolved in DMF (125 μL) was added and the solution stirred overnight in the dark. The reaction mixture was dialysed (cellulose ester, MWCO 0.1-0.5 kD) with water (3×500 mL with water change at 2h, 4h and dialysed a further 2h). The solution was subjected to RP-FCC (Isolera One, SiliaSep™ C18 12 g cartridge) with a gradient from 0 to 100% MeOH in H₂O. The fractions were lyophilized to yield Sulfo-Cy5 4-amino-1-butanol (1.4 μmol, 8%) as a blue powder. UP LC-MS (waters) method: 5-70% acetonitrile in water, both contain 0.1% formic acid, 3 mins run; Calculated m/z 714.29 (MH⁺), Found m/z: 714.27; RT (min) 1.14. Purity: >95%.

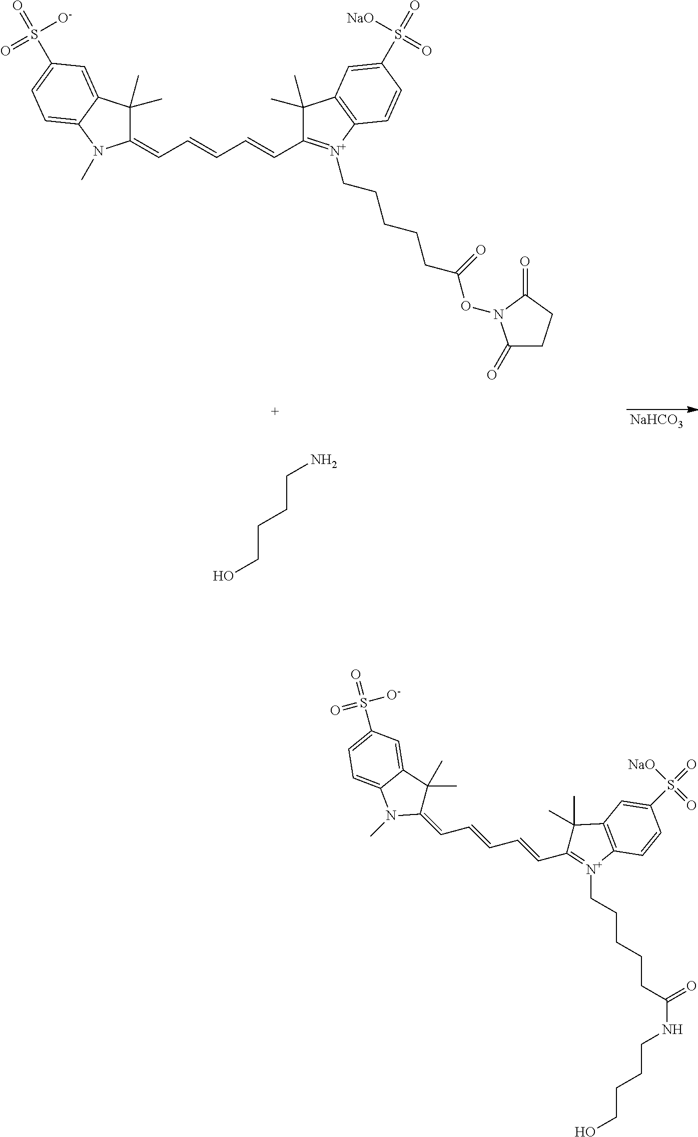

Stereoscopic, Microscopic, and Petrographic Analysis of Kidney Stones.

A total of 31 calculi were provided for stereoscopic gross examination. These stones represented multiple stone types including calcium oxalate, calcium phosphate, uric acid, struvite, brushite and cystine as confirmed by laboratory infrared spectroscopy. Whole calculi were submerged in solutions of 0.5 mM alendronate-fluorescein and notdronate-fluorescein and incubated for 30 minutes. The stones were subsequently washed in PBS and imaged using a fluorescent stereomicroscope.

For microscopic analysis, stone fragments were pulverized into a fine powder using a ceramic pestle and mortar. Approximately 10-20 mg of powder was suspended in 500 μL of distilled water, serving as a stock solution.

Two 25 μL samples of suspended calculi were further diluted in 225 μL of distilled water. Each aliquot was treated with 1 μL of 0.25 mM alendronate-fluorescein/notdronate-fluorescein for 15 minutes, and then centrifuged at 10,000×g's for one minute to pellet calculi fragments. The supernatant was discarded and the sample washed with distilled water three times to remove all unbound alendronate/notdronate. These samples were mounted onto slides and imaged using a fluorescent stereomicroscope.

For petrographic thin sectioning, whole calculi were embedded in epoxy resin and grinded down with water and silicone carbide. After grinding the calculi to a flat surface, calculi were mounted onto glass slides using epoxy resin. Excess stone material was cut off with a diamond blade cooled with Pella-A oil and the surface smoothened using a water-cooled diamond cup wheel. The sections were then hand rubbed with a glass plate using water and silicon carbide to achieve the desired thickness of 30 The sections were polished with Pella-A oil and were not sealed. Preliminary analysis of sections was carried out with Nikon TE200 microscope using polarized light and white light. The sections were then topically treated with 5 μL of 0.1 mM notdronate-fluorescein and incubated in dark for 15 minutes. Sections were thoroughly washed in phosphate buffered saline (PBS) to remove excess probe. Sections were then imaged in the same position with normal, polarized, and fluorescence excitation light. The same sections were then treated with 5 μL of 0.1 mM alendronate-fluorescein and imaged again.

Nanoscale Flow Cytometry of Urine Samples for Enumeration of Alendronate +ve Nanocrystals. 31 patient urine samples were analyzed using nanoscale flow cytometry. 10 μL of patient urine was diluted in 225 μL of PBS and then treated with 1 μL of 0.25 mM alendronate-fluorescein or notdronate-fluorescein as well as 1 μL of 0.25 mM alendronate-Cy5 or notdronate-Cy5. Samples were incubated for 15 minutes before being sonicated for an additional 15 minutes prior to analysis by nanoscale flow cytometry.

All flow cytometry data was collected with the Apogee A50-Micro Nanoscale Flow Cytometer.

Results

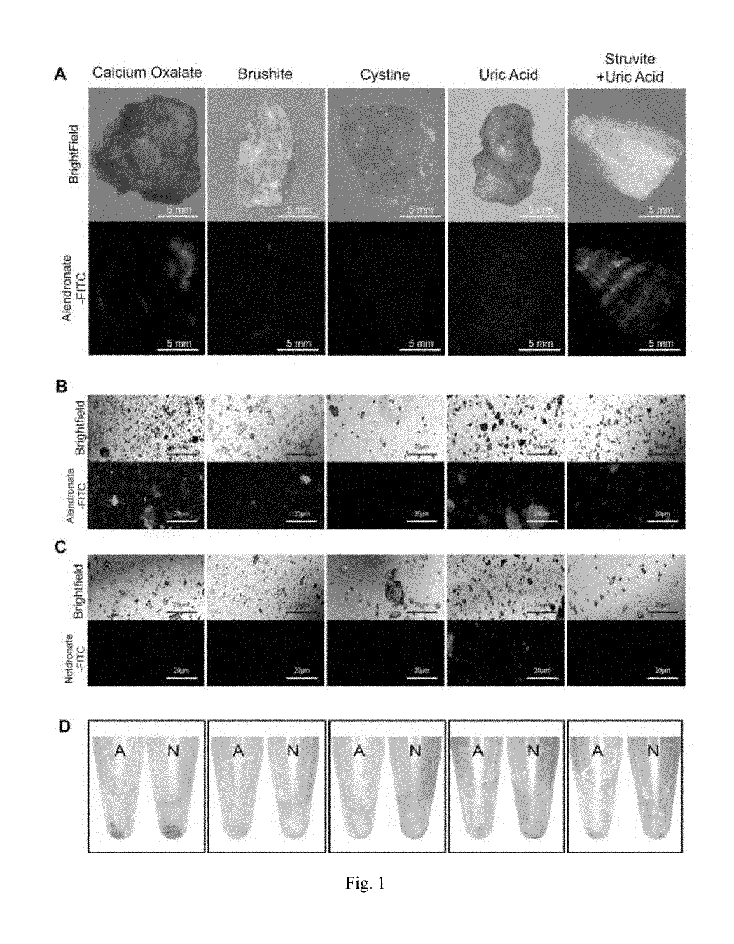

Stereoscopic Analysis of Calculi Stained with Fluorescent Alendronate Probes. Fluorescence-based probe analysis of renal stones at the macroscopic level produced inconclusive results; the majority of calcium oxalate calculi did not exhibit significant fluorescent signal when stained with alendronate-fluorescein but some calculi exhibited a patchy signal (FIG. 1A, far left panels). Other stone types also did not exhibit significant fluorescent signal, with the struvite+uric acid calculi revealing a striated fluorescent signal across the stone surface (FIG. 1A, far right panels). However, when all stone subtypes were pulverized and then stained with alendronate-fluorescein, fluorescent signal was clearly evident at the microscopic level for the calcium oxalate, brushite and struvite subtypes (FIG. 1B, far left, second from far left, far right panels respectively). Both cystine and uric acid calculi subtypes were negative when treated with fluorescent alendronate probes although in some uric acid stones minor auto-fluorescence was observed (FIG. 1B, second panel from far right) when treated with alendronate-fluorescein probe, notdronate-fluorescein probe, and in the absence of probe. When pulverized stones were treated with notdronate-fluorescein, no signal was observed except for minor auto-fluorescence signal in a subpopulation of uric acid calculi (FIG. 1C). During preparation of pulverized calculi for microscopic imaging, calcium oxalate and brushite calculi pellets also exhibited different affinity macroscopically for alendronate-Cy5 probe compared to notdronate-Cy5 isotype control probes (FIG. 1D, far left panels and second from far left panels) as evident by the appearance of blue discoloration of the pulverized calculi fragments.

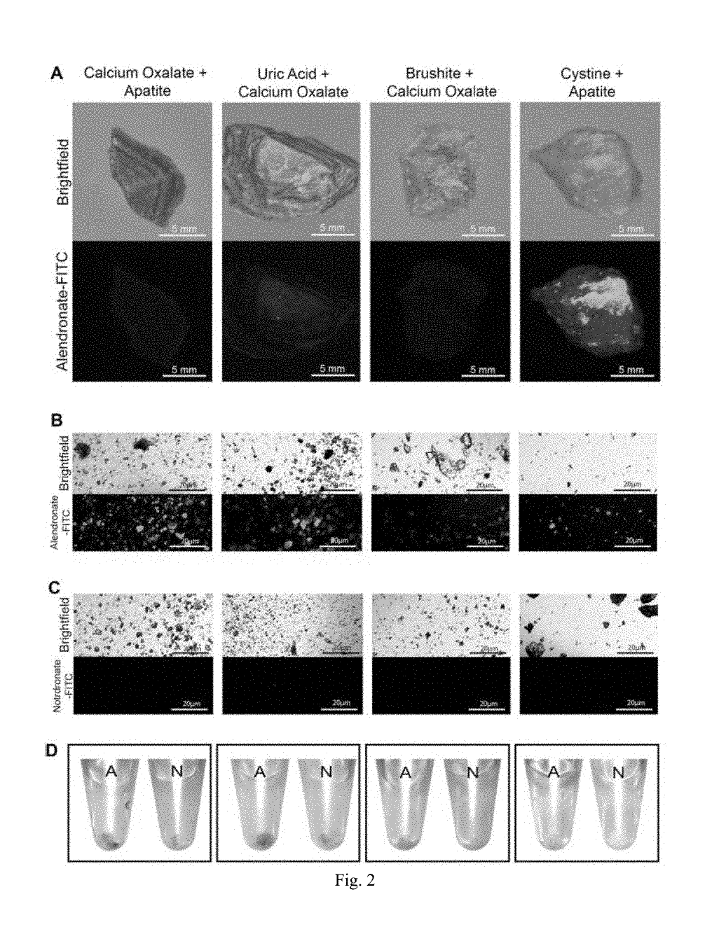

Many calculi are of mixed elemental composition and some of these calculi were submitted to staining with probes and evaluated as previously illustrated in FIG. 1. In FIG. 2A-C (far left panels), a single calcium oxalate+apatite calculus did not exhibit surface staining with alendronate probes but upon pulverization, did exhibit remarkable signal with microscopic and macroscopic analysis of calculi fragments compared to fragments stained with notdronate-fluorescein (FIG. 2A-D, far left panels). Other calculi subtypes (uric acid+calcium oxalate, brushite+calcium oxalate) also produced similar results (FIG. 2A-D, middle panels), while a cystine+apatite stone produced significant signal when imaged (FIG. 2A-D, far right panels).

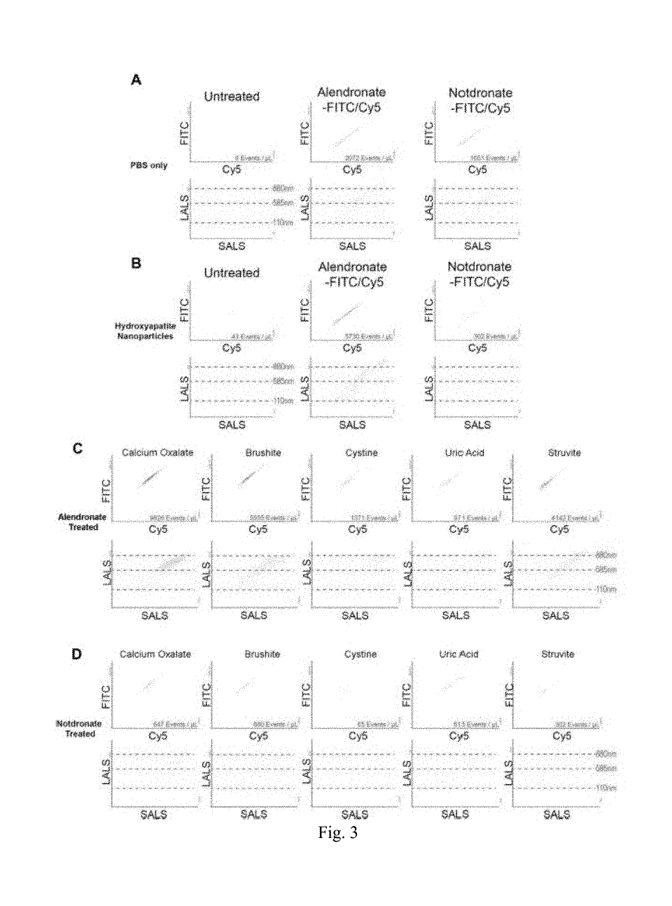

Nanoscale Flow Cytometry of Urine Samples for Alendronate +ve Nanocrystals. To evaluate the binding efficacy of fluorescent alendronate (alendronate-fluorescein or alendronate-Cy5) to nanocrystals released by calcium-positive calculi in patients, hydroxyapatite (HA) nanoparticles were used as a positive control and incubated with alendronate-fluorescein/Cy5 or notdronate-fluorescein/Cy5 probes when suspended in PBS, revealing specificity of the alendronate probes to HA nanoparticles compared to notdronate probes (FIG. 3A).

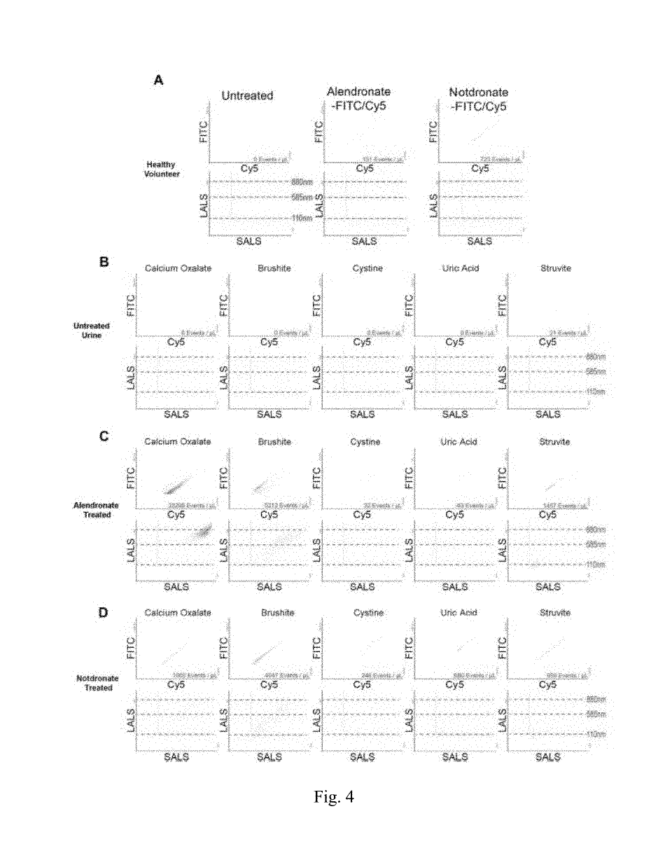

When pulverized calculi were re-suspended in PBS and treated with fluorescent alendronate probes, calcium oxalate samples generated the largest dual-positive fluorescent population followed by brushite and struvite samples. Pulverized uric acid and cystine calculi generated the lowest concentration of dual-positive events. The majority of stone samples when stained with notdronate probes produced a low number of dual-positive events (FIG. 3D). There was a lack of alendronate +ve nanocrystals in healthy volunteer urine (FIG. 4A). Analysis of healthy volunteer urine samples supplemented with pulverized stone subtypes showed no dual-positive events in the absence of alendronate-fluorescein/Cy5 probe (FIG. 4B) but significant dual-positive subpopulation in healthy volunteer urine supplemented with pulverized calcium oxalate, brushite, and struvite calculi (FIG. 4C) relative to notdronate controls (FIG. 4D).

Correlation of Calculi Nanocrystals in Urine and Clinical Stone Analysis.

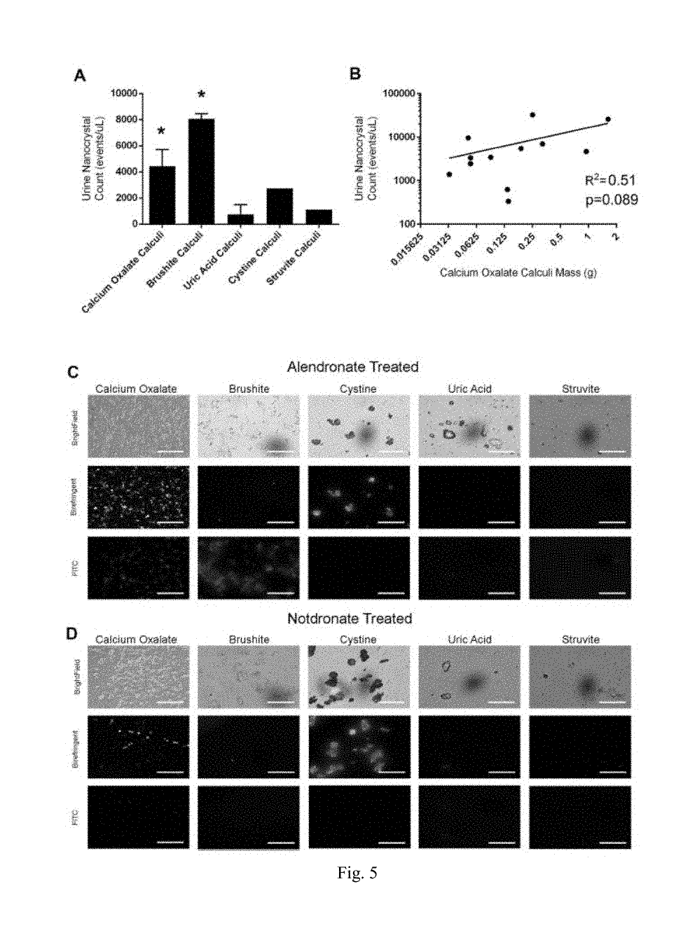

Enumeration of alendronate-fluorescein +ve events, known as calculi nanocrystals in patient urine samples was performed by nanoscale flow cytometry. Urine samples from calcium oxalate and brushite stone forming patients exhibited a statistically significant difference in dual-positive counts/μL compared to urine samples from healthy volunteers, and patients with uric acid calculi, struvite calculi, and cystine calculi (FIG. 5A). However, not all urine samples from patients with calcium oxalate calculi exhibited high nanocrystal counts, as a large range of alendronate-fluorescein +alendronate-Cy5 nanocrystal counts was observed in the calcium oxalate group and this was directly correlated to the weight of the calculus (FIG. 5B). This suggests that stone burden may be related to nanocrystal counts in patient urine. Microscopic analysis of urine samples confirmed the relative abundance of alendronate-fluorescein positive fragments in calcium oxalate and brushite subtypes (FIG. 5C, far left panels) relative to notdronate staining controls (FIG. 5D). Polarized light microscopy produced inconsistent results between urine samples representative of calculi subtypes.

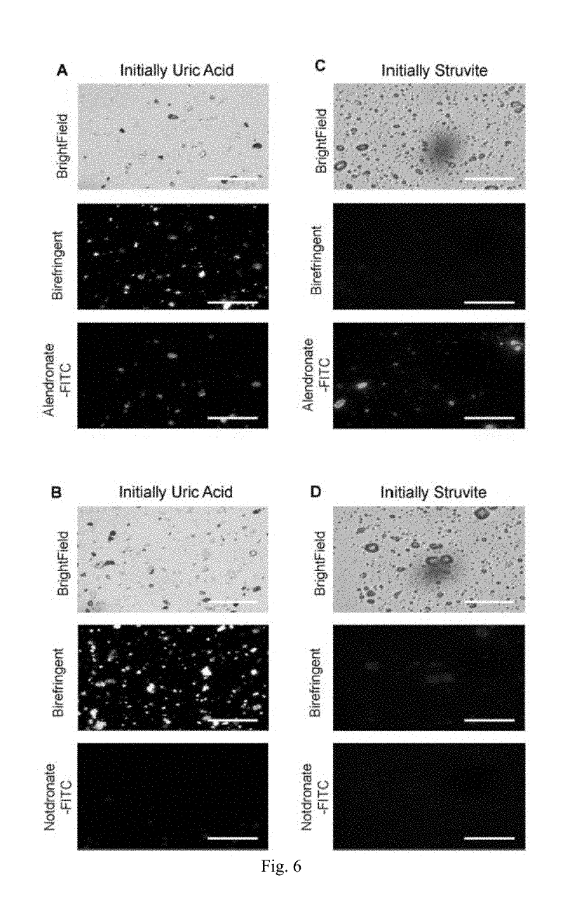

Urine from a patient with uric acid calculi exhibited high nanocrystal levels but when this entire calculus was pulverized for staining with alendronate-fluorescein and notdronate-fluorescein probes, microscopic fluorescently labelled fragments that were also birefringent under polarized light microscopy were observed indicating a calcium based composition (FIG. 6A,B). A similar urine sample from a struvite calculi patient also exhibited high nanocrystal counts and pulverized fragments bound the alendronate-fluorescein probe but did not exhibit birefringence signal (FIG. 6C,D).

Petrographic Thin Sections of Calculi Subtypes and Binding of Fluorescent Alendronate Probes.

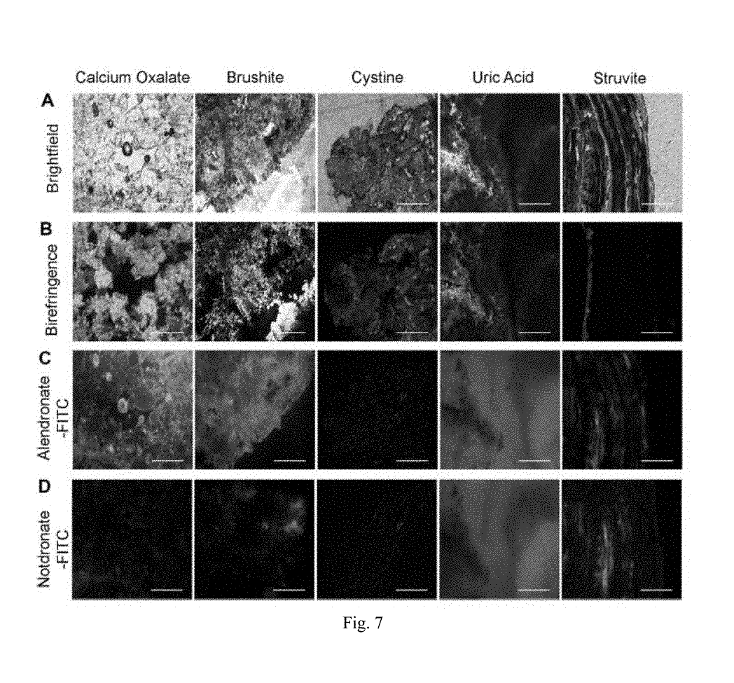

Prospectively collected paired sample sets of urine and calculi (N=31) were submitted to nanoscale flow cytometry and petrographic thin section staining respectively. Thin sections of stones were submitted to staining with alendronate-fluorescein and notdronate-fluorescein as a negative control. As anticipated, calcium oxalate stone petrographic thin sections resulted in abundant alendronate-fluorescein binding relative to notdronate-fluorescein staining whereas other thin sections of other calculi subtypes did not (FIG. 7). For example, uric acid calculi thin sections produced signal in both alendronate-fluorescein and notdronate-fluorescein stains, confirming an absence of calcium based crystals in this stone type. These results corroborated polarized light microscopy, which reveals “twinkling”-like spectral signal generated by calcium based and magnesium based crystals that make up calcium oxalate and struvite stones (FIG. 7B). Analysis of petrographic sections from calculi reveals a correlation between the number of nanocrystal events from urine obtained from nanoscale flow cytometry and the brightness or amount of twinkling obtained from polarized light microscopy.

We describe a non-invasive and high throughput means of evaluating stone burden and subtype by analyzing urine samples for the presence of nanocrystals that bind fluorescent alendronate probes. Despite the relatively small sample size for this initial study, we show that nanocrystal counts in urine correlate to calcium stone subtypes and also stone burden. These probes are able to identify patients with calcium oxalate and brushite calculi. In some cases, urine samples that had high nanocrystal counts from patients with struvite or uric acid calculi were reclassified when the stones were submitted to petrographic thin sectioning or pulverizing the calculi prior to staining with the fluorescent alendronate probes. Petrographic thin section analysis was important in revealing striations or deposits of calcium oxalate missed by mass spectrometry of the calculi surface or surface fragment thought to be representative of the entire calculi. Macroscopic analysis of whole renal calculi was determined to be a poor method of determining composition because the surface of many calculi are covered with biological material encasing a mineralized interior. When pulverized, fragments of calculi representing the interior were amenable to fluorescent alendronate probe staining and analysis by optical microscopy, and although this technique does not offer prognostic information it may improve accuracy in stone subtype diagnosis. However, nanoscale flow cytometry can be used to infer calculi composition and burden in a quantitative manner with greater accuracy. Since only a small amount of urine is needed, serial analyses can be performed to enhance clinical follow-up for patients making lifestyle changes and also assessing response to treatments such as shock wave lithotripsy. Other advantages include its non-invasive nature, rapid analysis of samples and the low cost of reagents. The average time between acquisition of a urine sample and generation of results is 5-10 minutes. Nanoscale flow cytometry instrumentation is not widespread with the analysis of nanocrystals in urine samples specific to the model of instrument used as described in this report. However, the unique capabilities offered by this instrumentation may yield clinically relevant information not possible by any other current means.

We found that petrographic thin sections, due to their ability to examine the calculi in their entirety, are a very accurate method of determining composition in a number of ways and should be adopted as part of the full clinical workup of calculi analysis. Thin sections of calculi provided a global understanding of the heterogeneous mineralization present within each calculus. While more time consuming, improving diagnosis of stone subtype would improve clinical outcomes since more specific treatments could be provided knowing what stone subtype was being treated. Overall, we present a high-throughput technique of evaluating stone subtype and burden and a histology-based technique of evaluating intra-stone heterogeneity with both techniques exhibiting the potential to improve patient monitoring and treatment decisions.

### REFERENCES

1. Scales, C. D., Smith, A. C., Hanley, J. M. & Saigal, C. S. Prevalence of kidney stones in the United States. Eur. Urol. 62, 160-165 (2012).
2. Stamatelou, K. K., Francis, M. E., Jones, C. a., Nyberg, L. M. & Curhan, G. C. Time trends in reported prevalence of kidney stones in the United States: 1976-1994. Kidney Int. 63, 1817-1823 (2003).
3. Fulgham, P. F., Assimos, D. G., Pearle, M. S. & Preminger, G. M. Clinical effectiveness protocols for imaging in the management of ureteral calculous disease: AUA technology assessment. J. Urol. 189, 1203-1213 (2013).
4. Pfister, S. A. et al. Unenhanced helical computed tomography vs intravenous urography in patients with acute flank pain: Accuracy and economic impact in a randomized prospective trial. Eur. Radiol. 13, 2513-2520 (2003).
5. Smith, R. C., Verga, M., McCarthy, S. & Rosenfield, A. T. Diagnosis of acute flank pain: value of unenhanced helical CT. AJR. Am. J. Roentgenol. 166, 97-101 (1996).
6. Parmar, M. S. Kidney stones. BMJ 328, 1420-1424 (2004).
7. Hess, B., Hasler-Strub, U., Ackermann, D. & Jaeger, P. Metabolic evaluation of patients with recurrent idiopathic calcium nephrolithiasis. Nephrol. Dial. Transplant 12, 1362-8 (1997).
8. Parks, J. H., Goldfisher, E., Asplin, J. R. & Coe, F. L. A single 24-hour urine collection is inadequate for the medical evaluation of nephrolithiasis. J. Urol. 167, 1607-12 (2002).
9. Viprakasit, D. P., Sawyer, M. D., Herrell, S. D. & Miller, N. L. Changing composition of staghorn calculi. J. Urol. 186, 2285-90 (2011).
10. Mostafavi, M. R., Ernst, R. D. & Saltzman, B. Accurate determination of chemical composition of urinary calculi by spiral computerized tomography. J. Urol. 159, 673-5 (1998).
11. Marchini, G. S. et al. Absolute Hounsfield unit measurement on noncontrast computed tomography cannot accurately predict struvite stone composition. J. Endourol. 27, 162-7 (2013).
12. Hidas, G. et al. Determination of renal stone composition with dual-energy CT: in vivo analysis and comparison with x-ray diffraction. Radiology 257, 394-401 (2010).
13. Basiri, A., Taheri, M. & Taheri, F. What is the state of the stone analysis techniques in urolithiasis? Urol. J. 9, 445-54 (2012).
14. Krambeck, A. E. et al. Inaccurate reporting of mineral composition by commercial stone analysis laboratories: implications for infection and metabolic stones. J. Urol. 184, 1543-9 (2010).
15. Lojo Oliveira, L. & Torrijos Eslava, A. Treatment of Paget's disease of bone. Reumatol. Clin. 8, 220-4 (2012).
16. Cole, L. E., Vargo-Gogola, T. & Roeder, R. K. Bisphosphonate-functionalized gold nanoparticles for contrast-enhanced X-ray detection of breast microcalcifications. Biomaterials 35, 2312-21 (2014).
17. Skogen-Hagenson, M. J., Salzman, G. C., Mullaney, P. F. & Brockman, W. H. A high efficiency flow cytometer. J. Histochem. Cytochem. 25, 784-9 (1977).
18. Sippel, R. F. & Glover, E. D. Structures in Carbonate Rocks Made Visible by Luminescence Petrography. Science 150, 1283-7 (1965).
19. Ivarsson, M. Advantages of doubly polished thin sections for the study of microfossils in volcanic rock. Geochem. Trans. 7, 5 (2006).
20. Giannossi, M. L., Mongelli, G., Tateo, F. & Summa, V. Mineralogical and morphological investigation of kidney stones of a Mediterranean region (Basilicata, Italy). J. Xray. Sci. Technol. 20, 175-86 (2012).
21. Fernandez-Conde, M., Serrano, S., Alcover, J. & Aaron, J. E. Bone metaplasia of urothelial mucosa: an unusual biological phenomenon causing kidney stones. Bone 18, 289-91 (1996).

Future applications claiming priority to this application may or may not include the following claims, and may include claims broader, narrower, or entirely different from the following claims.

The above disclosure generally describes the present invention. Changes in form and substitution of equivalents are contemplated as circumstances may suggest or render expedient. Although specific terms have been employed herein, such terms are intended in a descriptive sense and not for purposes of limitation. Other variations and modifications of the invention are possible. As such modifications or variations are believed to be within the sphere and scope of the invention as defined by the claims appended hereto.

1. A method for detecting urinary calculi in a subject, the method comprising:
(a) exposing an urine sample of the subject to at least one labeled binding probe that is specific to calcium/magnesium-containing urinary calculi and (b) detecting urinary calculi bound to the at least one labeled binding probe.

2. The method of claim 1, wherein the detection of the at least one labeled binding probe is obtained with one or more of stereoscopic, microscopic and/or flow cytometry analysis of the urine sample.

3. The method of claim 1, wherein the detection of the at least one labeled binding probe is obtained with nanoscale flow cytometry.

4. A method for determining urinary calculi type in a subject, the method comprising:
(a) exposing a urine sample obtained from the subject to at least one labeled binding probe that is specific to calcium/magnesium-containing urinary calculi and s (b) detecting urinary calculi bound to the at least one labeled binding probe, wherein the detected urinary calculi bound to the at least one labeled binding probe are calcium based urinary calculi.

5. The method of claim 4, wherein the detection of the at least one labeled binding probe is obtained with one or more of stereoscopic, microscopic and/or flow cytometry analysis of the urine sample.

6. The method of claim 4, wherein the detection of the at least one labeled binding probe is obtained with nanoscale flow cytometry.

7. A method for measuring the amount urinary calculi in a subject, the method comprising (a) exposing a urine sample obtained from the subject to at least one labeled binding probe that is specific to calcium/magnesium-containing urinary calculi, and (b) measuring the amount of urinary calculi bound to the at least one labeled binding probe.

8. The method of claim 7, wherein the measured amount of urinary calculi bound to the at least one labeled binding probe is then compared with a reference value and a diagnosis of the subject is obtained based on results of the comparison.

9. The method of claim 7, wherein the measurement of calculi bound to the at least one labeled binding probe is obtained by flow cytometry.

10. The method of claim 7, wherein the measurement of calculi bound to the at least one labeled binding probe is obtained by nanoscale flow cytometry.

11. The method of claim 7, wherein the method is for monitoring a therapeutic treatment to reduce or eliminate urinary calculi in the subject, wherein the method for monitoring the therapeutic treatment comprises: (a) measuring the amount of urinary calculi in a first urine sample obtained from the subject at a first time point, the measuring comprising:
i) exposing the first urinary sample to at least one labeled binding probe that is specific to calcium/magnesium-containing urinary calculi; and
ii) measuring the amount of urinary calculi bound to the at least one labeled binding probe;
(b) measuring the amount of urinary calculi in a second urine sample obtained from the subject at a subsequent time point, the measuring comprising:
i) exposing the second urine sample to at least one labeled binding probe that is specific to calcium/magnesium-containing urinary calculi; and
ii) measuring the amount of urinary calculi bound to the at least one labeled binding probe in the second urine sample;
(c) comparing the measured urine calculi bound to the at least one labeled binding probe in the second urine sample with the measurement obtained in step (a) (ii); and
(d) detecting increased or reduced urinary calculi in the subject when there is a difference in the measurement obtained in step (a) (ii) relative to the measurement obtained in step (b) (ii), thereby monitoring therapeutic treatment based on said difference.

12. The method of claim 11, wherein the measurement of calculi bound to the at least one labeled binding probe is obtained by flow cytometry.

13. The method of claim 11, wherein the measurement of calculi bound to the at least one labeled binding probe is obtained by nanoscale flow cytometry.

14. The method of claim 1, wherein the method is to determine the susceptibility of the subject for kidney stones, wherein detection of calcium-containing particles in the urine of the subject is indicative of the subject's susceptibility for kidney stones.

15. The method of claim 14, wherein the detection of the at least one labeled binding probe is obtained with one or more of stereoscopic, microscopic and/or flow cytometry analysis of the urine sample.

16. The method of claim 14, wherein the detection of the at least one labeled binding probe is obtained with nanoscale flow cytometry.

17. The method of claim 1, wherein the at least one labeled binding probe includes detectably-labeled Alendronate.

18. The method of claim 1, wherein the label includes a fluorophore.

19. The method of claim 18, wherein the fluorophore is FITC or Cy5.

20. The method of claim 11, wherein the at least one labeled binding probe includes detectably-labeled Alendronate.
